(19) United States
(12) Patent Application Publication
(10) Pub. No.: US 2026/0266720 A1
(43) Pub. Date: Sep. 10, 2026

(54) Device and System for Non-Invasively Determining a Concentration of an Analyte

(71) Applicant: UNIVERSITY OF LEEDS, Leeds (GB)

(72) Inventors: Gin JOSE, Leeds (GB); Almut BEIGE, Leeds (GB); Benjamin DAWSON, Leeds (GB); Nicholas FURTAK-WELLS, Leeds (GB); Robert MATHIESON, Leeds (GB)

(21) Appl. No.: 19/167,566

(22) PCT Filed: Mar. 21, 2024
(86) PCT No.: PCT/GB2024/050766

§ 371 Date: Sep. 22, 2025

(30) Foreign Application Priority Data
Mar. 23, 2023 (GB) 2304226.0

(51) Int. Cl. G01N 21/64; A61B 5/145; A61B 5/1455; G01N 33/14

(52) U.S. Cl. CPC G01N 21/6408; A61B 5/14532; A61B 5/1455; G01N 33/143; G01N 33/146

(57) ABSTRACT

A device (100) to non-invasively determine a concentration of an analyte in a sampling volume (126) has a pulsed light source (114). A doped glass (104) has dopants that can emit fluorescence in a fluorescence wavelength range that at least partially overlaps with an absorption wavelength range of the analyte. The fluorescence has a first temporal fluorescence emission distribution in response to optical excitation by the pulsed light source (114) when the analyte concentration in the sampling volume is zero. A semi-transparent mirror (108) is interposed between the doped glass (104) and the sampling volume (126), such that the dopants emit fluorescence having a second temporal fluorescence emission distribution that is a function of the concentration of the analyte in the sampling volume (126). A detector (120) is configured to measure the second temporal fluorescence emission distribution such that the concentration of the analyte in the sampling volume (126) can be determined based on a change between the first temporal fluorescence emission distribution and the second temporal fluorescence emission distribution.

### FIELD OF THE INVENTION

The invention relates to a device for non-invasively determining a concentration of an analyte in a sampling volume. The invention also relates to a system comprising the device and a processor configured to determine the concentration of the analyte in the sampling volume.

### BACKGROUND

It would be beneficial to be able to measure a concentration of a particular atom or molecule of interest (which may be referred to as an analyte) in a substance in a non-invasive manner (i.e., without breaking the surface or container encapsulating the analyte).

For example, people with diabetes often need to regularly monitor levels of glucose in their blood to adjust the amount of insulin they need to take accordingly. This glucose monitoring is traditionally done through finger-prick testing, which involves puncturing the skin surface, often several times daily. So, it would be desirable to find a non-invasive way to monitor levels of glucose in the blood, which does not require repeated and uncomfortable puncturing of the skin.

In another example, is it also useful to be able to measure a concentration of an analyte of interest in a substance in a sealed container, such as a bottle or a jar, for example, determining the alcohol by volume of wine in a corked wine bottle, or whether a jar of manuka honey is authentic by measuring the concentration of methylglyoxal (MGO) or dihydroxyacetone (DHA). However, in order to measure the concentration of the analyte, it is typically necessary to break the seal of the container, e.g., by uncorking a bottle or removing the lid of a jar, thereby comprising any protective atmosphere inside the container and risking contamination of the product therein.

Additionally, attempting to measure the concentration of the analyte non-invasively using conventional spectroscopic techniques can provide inaccurate results in situations where containers, such as wine bottles and jars, are made from coloured glass (which is often the case), as the colouring of the glass affects the absorption and emission of light.

It is possible to determine a concentration of an analyte by exciting a dopant that has a fluorescence spectrum that overlaps with an absorption band of the analyte, measuring how the fluorescence lifetime of the dopant changes in response to the concentration of the dopant present in the vicinity of the analyte. Specifically, the spontaneous decay rate of the dopant changes when in the presence of the analyte in response to non-radiative dipole-dipole coupling between the analyte and the dopant caused by Foerster Resonance Energy Transfer (FRET). The concentration of the analyte can be inferred from the change between the spontaneous decay rate of the dopant in the presence of analyte and the spontaneous decay rate of the dopant in the absence of the analyte.

However, a drawback is that the efficiency of this energy transfer strongly decreases with increasing distance between the dopant and the analyte. Therefore, for a measurable effect on the fluorescence lifetime or the spontaneous decay rates of the dopant to be observed, the distance between the dopant and the analyte needs to be of the order of the wavelength of the emitted fluorescence from the dopant. This means the dopant and the analyte essentially need to be in direct contact with one another. As a result, it is very difficult to be able to determine the concentration of the analyte in a sampling volume (such as a person's finger or a container), in a non-invasive manner (without breaking the skin or opening the container).

It would be advantageous to improve upon the above-mentioned drawbacks by providing a way to non-invasively measure the concentration of an analyte in a sampling volume.

### SUMMARY

According to a first aspect of the invention, there is provided a device to non-invasively determine a concentration of an analyte in a sampling volume. The device comprises a pulsed light source and a doped glass comprising dopants that are configured to emit fluorescence (and/or photoluminescence). The fluorescence has a fluorescence wavelength range that at least partially overlaps an absorption wavelength range of the analyte. The fluorescence has a first temporal fluorescence emission distribution in response to optical excitation by the pulsed light source when the analyte concentration in the sampling volume is zero.

The device also comprises a semi-transparent mirror (which may also be referred to as a partially-transparent mirror) interposed between the doped glass and the sampling volume, such that the dopants emit fluorescence having a second temporal fluorescence emission distribution that is a function of the concentration of the analyte in the sampling volume. The device also comprises a detector configured to measure the second temporal fluorescence emission distribution such that the concentration of the analyte in the sampling volume can be determined based on a change between the first temporal fluorescence emission distribution and the second temporal fluorescence emission distribution.

In the presence of the semi-transparent mirror, atomic interactions between the dopant and the analyte do not occur only over distances on the order of the wavelength of the fluorescence emitted by the excited dopants but can occur even when separation of the dopant and the analyte is several orders of magnitude larger than the interaction range of free-space dipole-dipole interactions. In other words, it is no longer necessary for the dopant and the analyte to be essentially in direct contact with one another, as in the prior art. Instead, the concentration of an analyte in a sampling volume can be deduced even when the separation of the dopant and the analyte is orders of magnitude larger than the wavelength of the fluorescence emitted by the excited dopants, allowing for the concentration of the analyte to be determined in a non-invasive manner. As such, the invention finds potential applications in a wide range of fields, for example determining a glucose or alcohol concentration in a user's bloodstream without needing to break the user's skin. Additionally, in the case of an analyte contained within a container, the invention allows for a concentration of the analyte to be determined without breaking or opening the container, with wide ranging applications such as: determining properties of foodstuffs; chemical process monitoring; and detection of pollutants, bacteria and drinking water quality in environmental monitoring.

Optionally, the concentration of the analyte in the sampling volume can be determined based on a change between:
a baseline spontaneous emission rate probability distribution based on the first temporal fluorescence emission distribution; and
an analyte-induced spontaneous emission rate probability distribution based on the second temporal fluorescence distribution.

Optionally, the change between the baseline spontaneous emission rate probability distribution and the analyte-induced spontaneous emission rate probability distribution can be based on either:
an average of the analyte-induced spontaneous emission rate probability distribution and an average of the baseline spontaneous emission rate probability distribution; or
a broadening of the analyte-induced spontaneous emission rate probability distribution compared to the baseline spontaneous emission rate probability distribution.

Optionally, the doped glass and the sampling volume are each located at a distance from the semi-transparent mirror that is greater than a maximum wavelength of the fluorescence wavelength range. For example wherein the distance is in a range of 1 to 100,000 times greater than the maximum wavelength. In another example, the distance is in a range of 10 to 10,000 times greater than the maximum wavelength. As an example, the maximum wavelength may be 1500 nm and the distance may be in the millimetre range, allowing for the concentration of the analyte in the sampling volume to be determined non-invasively.

Optionally, the doped glass and the sampling volume are each located a substantially equal distance from the semi-transparent mirror. In this way, targeted mirror-mediated interactions between dopants in the doped glass and analytes in the sampling volume are enhanced.

Optionally, the device further comprises a spacer interposed between the semi-transparent mirror and the sampling volume. By controlling a thickness of the spacer, the doped glass can be located at a substantially equal distance from the semi-transparent mirror to the sampling volume, allowing for targeted mirror-mediated interactions between dopants in the doped glass and analytes in the sampling volume. For example, the spacer may have a thickness that corresponds with a desired sampling depth in the sampling volume.

Optionally, a first optical efficiency between the semi-transparent mirror and the sampling volume is different to a second optical efficiency between the semi-transparent mirror and the dopant. In this way, constructive interference on one side of the semi-transparent mirror will not cancel out destructive interference on the other side, thereby ensuring that the concentration of the analyte in the sampling volume can be reliably determined.

Optionally, the device comprises an optical element interposed between either the sampling volume and the semi-transparent mirror or between the semi-transparent mirror and the dopant in order to make the first optical efficiency different to the second optical efficiency.

Optionally, the optical element comprises: a light absorbing component; a light absorbing layer on a surface of the semi-transparent mirror; or a roughened surface of an optical component.

Optionally, the analyte is a glucose molecule, and the dopants are erbium ions. As fluorescence emitted by the erbium ions overlaps with an absorption band of the glucose molecule, the device can specifically measure a concentration of glucose molecules, for example in a person's blood or in a fluid in a container.

Optionally, the analyte is an alcohol molecule, and the dopants are thulium ions. As fluorescence emitted by the thulium ions overlaps with an absorption band of the alcohol molecule, the device can specifically measure a concentration of alcohol molecules, for example in a person's blood or in a fluid in a container (e.g., an alcoholic beverage).

Optionally, the doped glass further comprises a sensitizer. The sensitizer may comprise ions that are chosen to strongly absorb light from the pulsed light source and non-radiatively transfer excitation energy to the dopants in the doped glass, thereby enhancing the fluorescence emitted by the dopants in the doped glass. The sensitizer may comprise ytterbium ions.

Optionally, the analyte is one of a blood analyte or a foodstuff analyte.

Optionally, the analyte is a blood analyte, and the detector further comprises a contact surface for receiving a skin surface having the blood analyte located at a subcutaneous depth, wherein the semi-transparent mirror is disposed between the contact surface and the doped glass. In this way, a concentration of the blood analyte can be reliably and repeatably determined as the skin surface is positioned at a consistent distance from the semi-transparent mirror each time a measurement is performed.

Optionally, the device further comprises a processor configured to determine the concentration of the analyte in the sampling volume based on the change between the first temporal fluorescence emission distribution and the second temporal fluorescence emission distribution.

Optionally, the device further comprises means for transmitting the second temporal fluorescence emission distribution to a remote server for determining the concentration of the analyte in the sampling volume. In this way, the computational complexity of the device is reduced.

According to a second aspect of the invention, there is provided a system comprising the device of the first aspect and a processor configured to determine the concentration of the analyte in the sampling volume based on the change between the first temporal fluorescence emission distribution and the second temporal fluorescence emission distribution.

Optionally, the processor is located on the device or on a server remote from the device.

### BRIEF DESCRIPTION OF THE DRAWINGS

The invention will now be described, by way of example only, with reference to the accompanying drawings, in which:

FIG. 1 is a schematic view of a device for non-invasively determining a concentration of an analyte in a user's body part;

FIG. 2 is a graph of an emission profile of erbium ions overlaid onto an absorption spectrum of glucose molecules;

FIG. 3a illustrates dipole interactions between dopants in the absence of analyte;

FIG. 3b illustrates a fluorescence decay curve of a dopant in response to a laser pulse in the absence of analyte;

FIG. 3c illustrates a spontaneous emission rate probability distribution in the absence of analyte;

FIG. 4a illustrates a fluorescence decay curve of a dopant in response to a laser pulse in the presence of analyte;

FIG. 4b illustrates an analyte-induced spontaneous emission rate probability distribution;

FIG. 5a illustrates interactions between dopants and analyte when a semi-transparent mirror and a spacer is placed therebetween;

FIG. 5b illustrates interactions between dopants and analyte when a semi-transparent mirror, but no spacer, is placed therebetween;

FIGS. 6a to 6c are illustrative diagrams showing interference patterns formed by two interacting atoms positioned either next to one another or placed on opposing sides of a semi-transparent mirror;

FIG. 7 is a schematic view of a device for non-invasively determining a concentration of an analyte in a sampling volume at a depth below a surface of the sampling volume;

FIG. 8a is a graph of intensity vs time for spontaneous decay of an excited dopant; and

FIG. 8b is a graph of showing divergence of a spontaneous decay function for an excited dopant.

In the drawings, like parts are denoted by like reference numerals.

### DETAILED DESCRIPTION

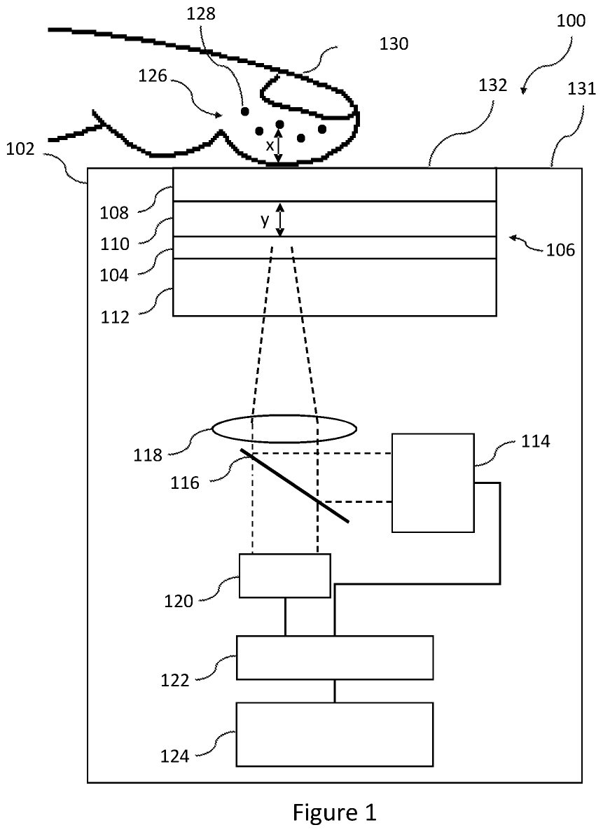

FIG. 1 is a schematic of a device 100 for non-invasively determining a concentration of an analyte 128, such as glucose, in a user's bloodstream. A user's finger 130, or another body part such as a wrist, is placed on contact surface 132 allowing the device 100 to measure the concentration of the analyte 128 in a sampling volume 126 located subcutaneously within the user's finger 130.

The device 100 has a housing 102 with a user-facing outermost surface 131 that contains the contact surface 132. Extending from the contact surface 132 into the interior of the housing 102 is a component stack 106 which has a number of optical components arranged in a layered fashion. The component stack 106 has a semi-transparent mirror 108. By “semi-transparent”, we mean that the semi-transparent mirror 108 is at least partially transparent. The contact surface 132 is either a surface of the semi-transparent mirror 108 itself (in which case the semi-transparent mirror 108 forms part of the user-facing outermost surface 131 of the housing 102), or a protective (for example, glass) layer disposed over the semi-transparent mirror 108.

The component stack 106 also has a doped layer 104 located under the semi-transparent mirror 108. The doped layer 104 contains dopants which are deposited onto a glass substrate 112 (such as silica glass) or dopants that are diffused into the glass substrate 112 (for example, as described in WO 2013/117941 A2, which is incorporated herein by reference). The thickness of the doped layer 104 is typically around 1 micron.

There is a gap between the semi-transparent mirror 108 and the doped layer 104 formed by an optically transparent spacer 110. The thickness of the spacer 110 is such that the doped layer 104 and the sampling volume 126 containing analytes 128 in the user's finger 130 are at substantially equal distances (x and y) from opposing sides of the semi-transparent mirror 108 (see FIG. 1). Instead of the gap being formed by a spacer 110 made from an optically transparent material (such as glass), the gap could instead be formed by an air gap. Alternatively, the thickness of the optical components in the component stack 106 (e.g., the semi-transparent mirror 108) may provide a sufficient gap if the sampling volume 126 is near the surface of the user's finger 130 in contact with the contact surface 132, such that no specific gap or spacer 110 is required.

The housing 102 of the device 100 further contains a pulsed light source 114, a beam splitter 116 and a light detector 120. The pulsed light source 114 (e.g., a laser diode) emits light pulses which the beam splitter 116 reflects towards the doped layer 104. The light pulses cause the dopant in the doped layer 104 to emit fluorescence. Fluorescence emitted by the dopant is transmitted through the beam splitter 116 and is collected by the light detector 120. The skilled person would appreciate that emission of fluorescence by the dopant could also include the emission of other forms of photoluminescence (e.g., phosphorescence). It would also be appreciated that the light pulses may excite a first dopant in the doped layer 104 which may act as a sensitiser, i.e., rather than emitting fluorescence itself, this first dopant/sensitiser non-radiatively transfers the excitation energy to a second dopant that emits fluorescence.

Optionally, a lens 118 may be interposed between the beam splitter 116 and the glass substrate 112 to generate a suitably sized light spot on the doped layer 104 (as shown by FIG. 1). However, the skilled person would appreciate that other lenses and optical elements may be required in the beam path based on the optical characteristics of the components of the device 100, such as the size and divergence of the pulsed light source 114, detection area of the light detector 120, and the dimensions of the sampling volume 126.

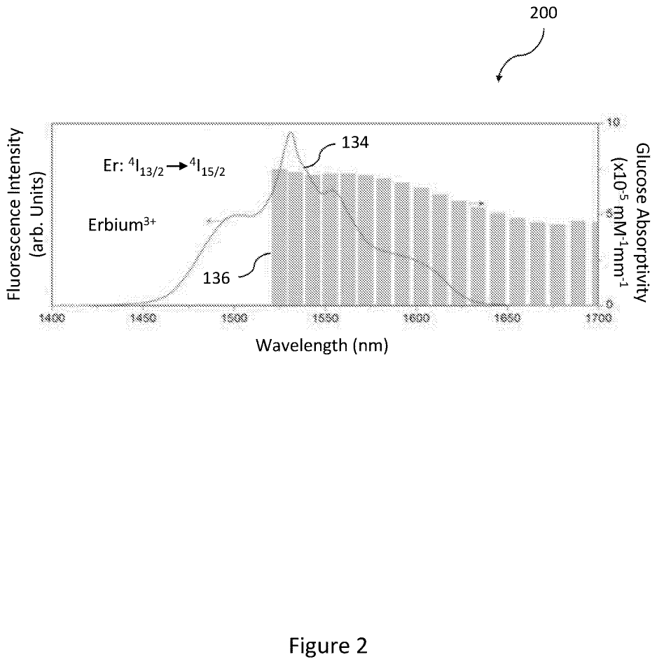

By carefully choosing the dopants in the doped layer 104, the device 100 can be tailored to specifically determine the concentration of a particular analyte 128. Continuing the example above where the analyte 128 is glucose in a user's finger 130, erbium ions are a suitable choice for the dopants in the doped layer 104. FIG. 2 is a graph 200 plotting fluorescence intensity 134 of erbium Er³⁺ ions (dopants) as a function of wavelength overlaid onto absorptivity 136 of glucose molecules (analytes) as a function of wavelength.

As can be seen in FIG. 2, fluorescence emitted by the erbium ions overlaps with an absorption band of the glucose molecules, causing spontaneous emission rates of the erbium ions to change compared to spontaneous emission rates of the erbium ions in the absence of glucose molecules. As such, by selecting erbium ions as the dopants in the doped glass 104, the device 100 can specifically measure a concentration of glucose molecules, rather than other biomarkers or analytes present in the skin or blood. The data for the absorptivity 136 of the glucose molecules as shown by FIG. 2 was determined by AMEROV, AK, Applied Spectroscopy, October 2004, Vol. 58, Issue 10, Pages 1195-1204.

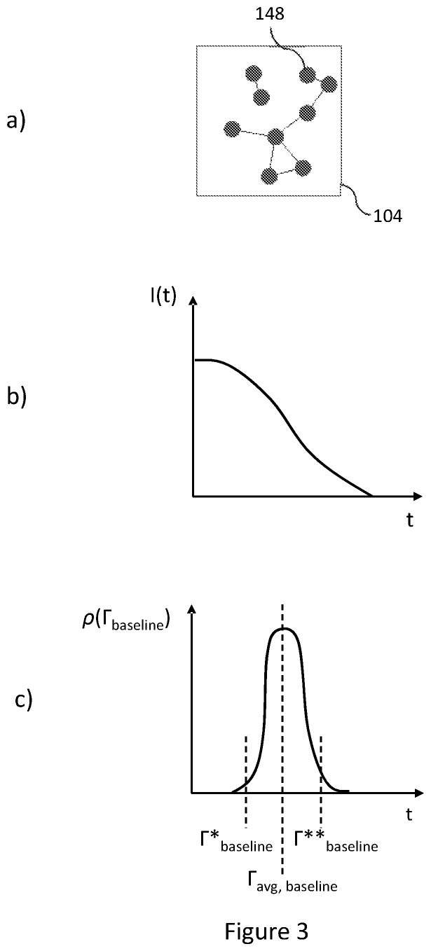

In order to determine how the presence of glucose molecules in the user's finger 130 influences the spontaneous emission rates of the fluorescence from the erbium ions in the doped layer 104, it is first necessary to establish a baseline for how the erbium ions behave when excited by the light source 114 in the absence of glucose molecules, which will now be discussed in relation to FIGS. 3a-3c.

In the absence of glucose molecules, erbium ions 148 experience dipole-dipole interactions (and potentially other atomic interactions) with neighbouring erbium ions (these interactions are indicated by connecting lines between erbium ions 148 in FIG. 3a), along with interactions with other nearby atoms, ions and molecules other than erbium that are present in the doped layer 104. These interactions modify the fluorescence characteristics (fluorescence lifetime and spontaneous emission rate) of the doped layer 104 compared with isolated erbium ions 148. Therefore, it is necessary to measure the fluorescence characteristics of the erbium ions 148 in the doped layer 104 in order to be able to calculate the concentration of the analyte. It is important that the fluorescence characteristic of the erbium ions 148 in the doped layer 104 is determined in the absence of analyte which could otherwise modify the fluorescence characteristics of the erbium ions 148. The fluorescence characteristics of the erbium ions 148 in the doped layer 104 in the absence of analyte can then be used as a baseline for establishing the change in fluorescence characteristics of the erbium ions 148 that the presence of the analyte brings about.

To establish the fluorescence characteristics of the erbium ions 148, temporal fluorescence distributions of the erbium ions 148 are recorded. The erbium ions 148 are excited by light pulses from the light source 114. In between light pulses, the intensity I of the fluorescence emitted by the erbium ions 148 is recorded as a function of time t at the light detector 120 (FIG. 3b). By averaging I(t) of the fluorescence emitted by the erbium ions 148 over a number of erbium ions 148 we can arrive at a spontaneous emission rate probability distribution p(Γ) for the erbium ions 148 in the doped layer 104.

Initially, a first (or baseline) temporal fluorescence distribution is measured for the doped layer 104 in the absence of analyte to establish a baseline spontaneous emission rate probability distribution p(Γ_{baseline}), as illustrated by FIG. 3c.

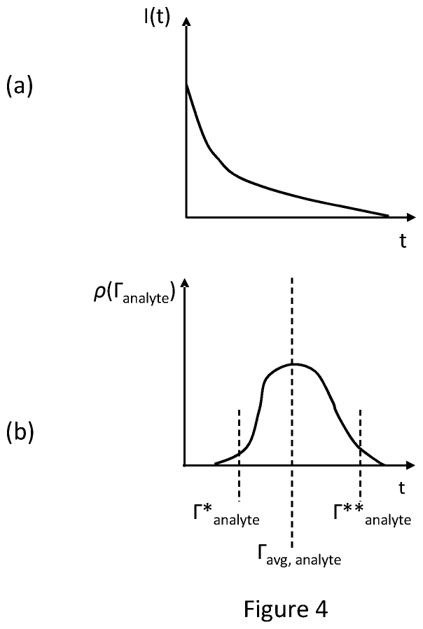

Once this baseline has been established, a second (or analyte-induced) temporal fluorescence distribution is determined for the doped layer 104 in the presence of analyte to determine an analyte-induced spontaneous emission rate probability distribution p(Γ_{analyte}) for the doped layer 104, as illustrated by FIG. 4b.

As can be seen by comparing FIGS. 3c and 4b, the presence of analyte changes the analyte-induced spontaneous emission rate probability distribution p(Γ_{analyte}) compared with the baseline spontaneous emission rate probability distribution p(Γ_{baseline}). This change, which can be used to infer the concentration of the analyte, can be characterised by comparing a number of statistical parameters between the analyte-induced spontaneous emission rate probability distribution p(Γ_{analyte}) compared with the baseline spontaneous emission rate probability distribution p(Γ_{baseline}). For example, comparing the change in the average spontaneous emission rates Γ_{avg,baseline} and Γ_{avg,analyte}. However, since the average spontaneous emission rates Γ_{avg,baseline} and Γ_{avg,analyte} may change only minimally, it is preferable to compare the width of the probability distributions which will change more strongly in response to the analyte-induced broadening. For example, the width between minimum and maximum threshold emission rates Γ* and Γ** can be determined for each probability distribution and compared to characterise the broadening. Details on how the minimum and maximum threshold emission rates Γ* and Γ** may be determined are provided below in the section entitled “Determination of Fluorescence Lifetime Measurements”.

The skilled person would expect such analyte-induced broadening only to be observed when the separation distance between the analyte molecules and the dopant are comparable to the wavelength of fluorescence emitted by the dopant (in other words, the glucose molecules and the erbium ions must be essentially adjacent to one another) because the dipole-dipole interactions between the analyte and dopant are strongly dependent on distance.

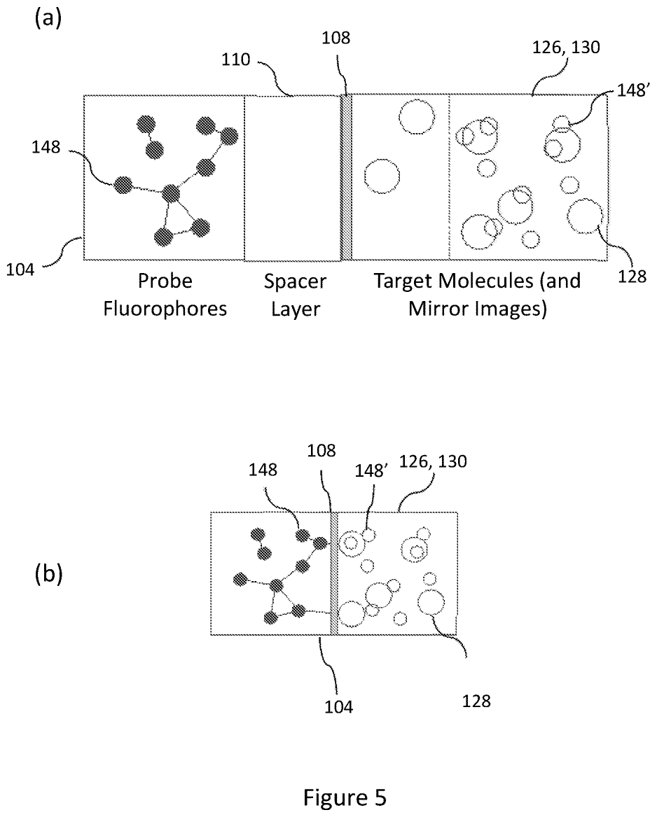

However, the inventors have surprisingly found that, by inserting the semi-transparent mirror 108 between the analyte and the dopant, the separation distance can be increased considerably (i.e., by many orders of magnitude longer than the wavelength of fluorescence emitted by the dopant). The inventors refer to this process as “mirror-mediated targeted remote interactions” between atoms (the physics of which is described in detail below at the sections entitled “Determination of Fluorescence Lifetime Measurements” and “Annex”). By exploiting this process, the device 100 can target optical measurements to non-invasively determine a concentration of an analyte at a depth in a sampling volume. For example, the device 100 can determine a glucose molecule concentration at a subcutaneous depth in the user's finger 130 in a non-invasive manner, as described below with respect to FIGS. 5a and 5b. A proof-of-concept study showing that the device 100 is capable of producing clinically acceptable subcutaneous glucose concentration measurements in a pig skin model is provided below in Example 1.

### Ultra-Long-Range Mirror-Mediated Targeted Remote Atomic Interactions

The device 100 can be configured to determine analyte 128 concentrations at a particular distance away from the contact surface 132 of the device 100. Continuing with the example where the analytes of interest are glucose molecules in a user's finger 130, glucose molecules are typically more highly concentrated in the lower layers of the skin, i.e., in the dermis. It can therefore be useful to additionally incorporate a spacer layer, such as spacer layer 110 shown by FIG. 1, between the semi-transparent mirror 108 and the doped layer 104. Whilst there exists an intrinsic spacing between the analyte 128 and the dopants 148 due to the presence of the semi-transparent mirror 108 (see, e.g., FIG. 5b), incorporating the spacer layer 110 allows the device 100 to perform targeted analyte concentration measurements at a specific distance away from the contact surface 132 that is substantially the same as the thickness of the spacer 110. In this way, the measurement depth of the device 100 can be controlled by changing the thickness of the spacer layer 110 accordingly. For example, if it is desired to determine the concentration of the analyte at a sampling distance on the order of millimetres away from the contact surface 132, a suitably sized spacer layer 110 can be used having a corresponding thickness (i.e., millimetres). As the required sampling distance decreases, the thickness of the spacer layer 110 can be reduced accordingly and vice versa.

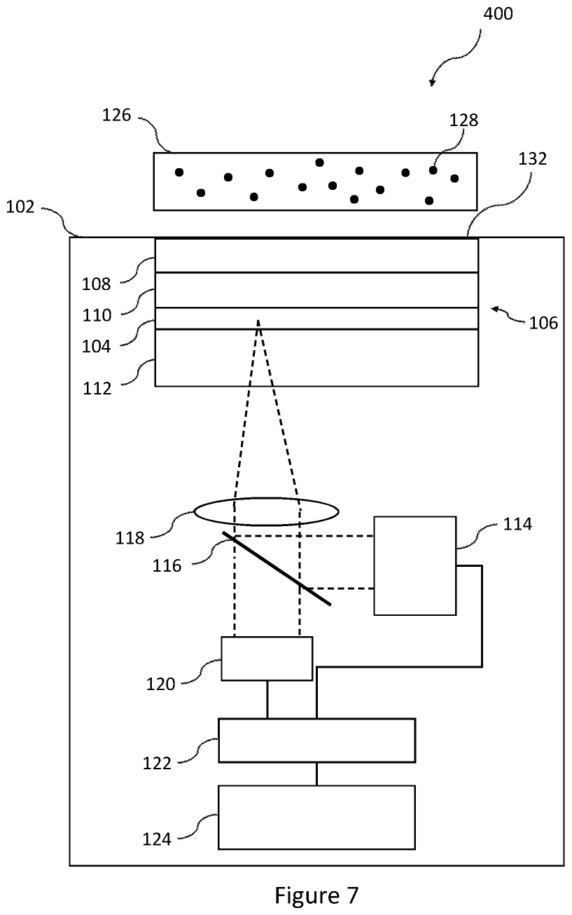

FIG. 5a is a simplified illustration showing part of the device 100, namely the doped layer 104, the semi-transparent mirror 108, and the spacer layer 110 of the device 100. The semi-transparent mirror 108 is placed between the spacer layer 110 and a medium (such as the user's finger 130, or the sampling volume 126 discussed in connection with FIG. 7 below) which contains target analytes 128, such as the glucose molecules. The purpose of the semi-transparent mirror 108 is to reduce an effective optical distance between the erbium ions 148 (represented by black circles in FIG. 5a) and the glucose molecules 128 (represented by the larger of the white circles in FIG. 5a). In the presence of the semi-transparent mirror 108, half of the light emitted by the excited erbium ions 148 is reflected by the semi-transparent mirror 108 and therefore appears to originate from behind the semi-transparent mirror 108, i.e., from mirror images of the erbium ions 148′ (represented by the smaller white circles in FIG. 5a).

Continuing the example above where the analytes 128 are glucose molecules and the dopants are erbium ions, the spacer layer 110 promotes interactions between the erbium ions 148 and the glucose molecules 128 only if the glucose molecules 128 and the erbium ions 148 are situated at a substantially equal distance from opposing sides of the semi-transparent mirror 108. As shown by FIG. 5a, the semi-transparent mirror 108 projects mirror-images of the erbium ions 148 to the side of the semi-transparent mirror 108 opposing the doped layer 104. The semi-transparent mirror 108 now promotes atomic interactions between mirror images 148′ of the erbium ions and the glucose molecules 108. Owing to the presence of the spacer layer 110, atomic interactions between the erbium ions 148 and their own mirror images 148′ become long-range and are therefore negligible. Any changes to atomic decay rates of the erbium ions, compared to the case above where no glucose molecules are present (see FIGS. 3a-c), are now only due to the presence of the glucose molecules, thus increasing the sensitivity of measurements of glucose molecule concentration. Ideally, the spacer layer 110 should have a thickness such that time taken for light to travel from the erbium ions to the semi-transparent mirror 108 remains small compared to the spontaneous emission rate of excited atomic states (e.g., of the erbium ions and excited electronic states of the glucose molecules). Typically, a spacer layer 110 thickness within the millimetre range is acceptable.

The concentration of glucose molecules at a particular subcutaneous depth in the user's finger 130 can be determined based on the same approach described in FIGS. 3 and 4.

### Close and Medium-Range Mirror-Mediated Targeted Remote Atomic Interactions

Whilst the example described above in connection with FIG. 5a outlined the ability to extend the sampling distance to millimetre ranges through the inclusion of an appropriately sized spacer layer 110 in the device 100, in cases where the sampling distance is on the order of microns (e.g., if the analyte of interest is substantially at or just below the contact surface 132) the spacer layer 110 may be omitted completely (see, e.g., FIG. 5b discussed below), owing, at least in part, to the intrinsic spacing between the analyte and the dopant due to the presence of the semi-transparent mirror 108.

FIG. 5b is a simplified illustration showing a portion of the device 100. Specifically, FIG. 5b shows the doped layer 104 and the semi-transparent mirror 108 of the device 100 as seen in FIG. 1. The spacer layer 110 is not essential for the example of FIG. 5b. The semi-transparent mirror 108 is placed between the doped layer 104 and a sampling volume 126 (such as the user's finger 130) which contains target analytes 128, such as the glucose molecules. As described above, the purpose of the semi-transparent mirror 108 is to reduce an effective optical distance between the erbium ions 148 (represented by black circles in FIG. 5b) and the glucose molecules 128 (represented by the larger of the white circles in FIG. 5b). In the presence of the semi-transparent mirror 108, half of the light emitted by the excited erbium ions 148 is reflected by the semi-transparent mirror 108 and therefore appears to originate from behind the semi-transparent mirror 108, i.e., from mirror images of the erbium ions 148′ (represented by the smaller white circles in FIG. 5b). The concentration of glucose molecules in the sampling volume 126 can be determined based on the same approach described in FIGS. 3 and 4.

The closer the erbium ions 138 are to the surface of the semi-transparent mirror 108 (i.e., where the spacer 110 is small or not present), the more complex it becomes to theoretically explain how their spontaneous emission rates change. This is because, in addition to interactions between the erbium ions 148 and the glucose molecules 128, interactions also occur between erbium ions 148 and their own mirror images 148′, which can partially mask the effect that the presence of the glucose molecules 128 has on the spontaneous emission rate of the erbium ions 148. However, the glucose molecule concentration can still be determined in the case of a spacer 110 that is small or not present as long as a glucose molecule concentration-dependent change in the spontaneous emission rate of the erbium ions 148 can be observed.

### Atomic Dipole-Dipole Interactions

The ability to determine a glucose molecule concentration at a subcutaneous depth in a user's finger 130, as introduced above with respect to FIGS. 5a and 5b, arises due to atomic dipole-dipole interactions between the erbium ions and the glucose molecules caused by interference effects.

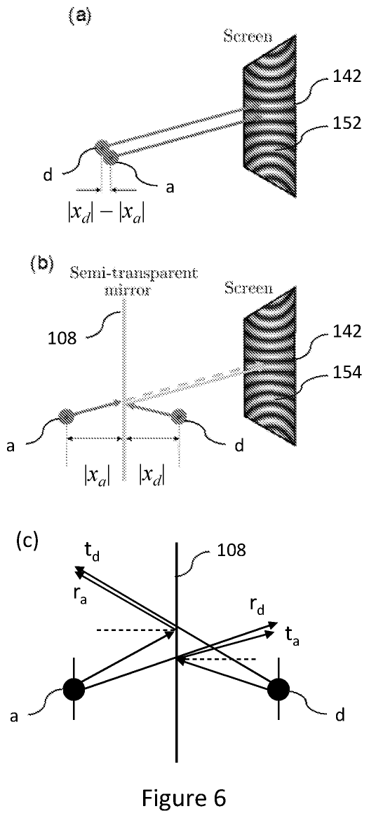

FIG. 6 conceptually illustrates interference effects between a dopant, d, and analyte, a, in the presence of the semi-transparent mirror 108 (FIG. 6b) and in the absence of the semi-transparent mirror 108 (FIG. 6a).

In FIG. 6a, dopant, d, and analyte, a, are spaced at a distance |x_{d}|-|xₐ| without a mirror 108 between them. If dopant, d, and analyte, a, are repeatedly excited by a train of light pulses (e.g., from light source 114) they emit photons at a constant rate. When the distance |x_{d}|-|xₐ| is comparable to the wavelength of light emitted by dopant, d, and analyte, a, a first interference pattern 152 forms on a far-away screen 142.

Suppose now that dopant, d, and analyte, a, are trapped on opposite sides of a two-sided semi-transparent mirror as shown in FIG. 6b (i.e., the semi-transparent mirror 108 shown in FIG. 1) and the far-away screen 142 collects their spontaneously emitted photons, as illustrated in FIG. 6b. Considering dopant, d, and analyte, a, as radiating dipoles, we see that light from dopant, d, (that is reflected from the semi-transparent mirror 108) and light from analyte, a, (that is transmitted through the semi-transparent mirror 108) have paths of equal length to a common point on the far-away screen 142.

When dopant, d, and analyte, a, are located at equal distances from either side of the semi-transparent mirror 108, a second interference pattern 154 is generated on the far-away screen 142 that is substantially the same as the first interference pattern 152 apart from reduced visibility (i.e., half of the light emitted from dopant, d, and analyte, a, is emitted away from the semi-transparent mirror 108 and is therefore neither reflected nor transmitted by/through the semi-transparent mirror 108 and thus does not contribute to any interference effects).

FIG. 6c illustrates components of light emitted by dopant, d, and analyte, a, that are reflected or transmitted by semi-transparent mirror 108. Specifically, dopant, d, emits light and a component r_{d} is reflected from the mirror 108 while a component t_{d} is transmitted by the mirror 108. Analyte, a, emits light and a component rₐ is reflected from the mirror 108 while a component tₐ is transmitted by the mirror 108.

Energy conservation implies that constructive interference on one side of the semi-transparent mirror 108 implies destructive interference on the other side of the semi-transparent mirror 108. To prevent constructive interference on one side of the semi-transparent mirror 108 cancelling out destructive interference on the other side, a first optical efficiency on one side of the semi-transparent mirror 108 can be tailored to be different to a second optical efficiency on the other side of the semi-transparent mirror 108. To make the first optical efficiency different to the second optical efficiency, the semi-transparent mirror 108 needs to be asymmetric so that transmission and reflection rates on both sides of the semi-transparent mirror 108 are not the same (i.e., r_{d} tₐ≠rₐ t_{d}). One way of achieving this is to have the medium one side of the semi-transparent mirror 108 more absorbing than the medium on the other side, which is usually the case with an analyte on one side and ions on the other, owing to their inherently different optical properties.

The skilled person would appreciate that there are other ways of making the first optical efficiency different to the second optical efficiency (if there is no difference) or enhancing an inherent difference between the first optical efficiency and the second optical efficiency. For example, an optical element may be interposed between either the glucose molecules 128 and the semi-transparent mirror 108 or between the erbium ions 148 and the semi-transparent mirror 108. The optical component could be one or more of: a light absorbing component; a light absorbing layer on a surface of the semi-transparent mirror 108; or a roughened surface of the optical component, such that transmission and reflection rates on opposing sides of the semi-transparent mirror 108 are not the same.

Whilst the above examples have been discussed in the context where the dopants are erbium ions and the analytes 128 are glucose molecules, the skilled person will appreciate that the device 100 can be tailored to determine the concentration of other analytes of interest by choosing appropriate dopants. All that is needed is to replace erbium as the dopant with other rare earth or transition metal ions, which then act as the dopant and dipole source. For example, other rare earth or transition metal ions besides erbium Er³⁺ that are capable of emitting electromagnetic radiation (e.g., Tm³⁺, Nd³⁺, Yb³⁺, Eu³⁺, Tb³⁺, Sm³⁺, etc) can be used as suitable dopants to determine concentrations of particular analytes of interest. As one illustrative example, if the analyte 128 of interest is instead an alcohol molecule, then thulium (Tm³⁺) is a suitable dopant since the 1800 nm emission wavelength of thulium Tm³⁺ overlaps with an absorption band of the alcohol molecule. As a further example, different emission wavelengths of rare earth or transition metals can be utilised to expand the range of different analytes that can be detected. For example, whilst FIG. 2 illustrates that the 1555 nm emission wavelength of erbium Er³⁺ overlaps with a glucose absorption band, other emission wavelengths of erbium may be used to detect other analytes than glucose molecules.

### Determining a Concentration of an Analyte in a Sampling Volume

Whilst the examples described above have been directed to determining a concentration of glucose molecules in a user's bloodstream, the device 100 can equally be used to determine a concentration of an analyte of interest in other kinds of sampling volume, such as a region inside a jar or bottle, where it is desirable to be able to non-invasively measure the concentration of the analyte, such as glucose or some other analyte.

FIG. 7 illustrates a device 400 configured to determine a concentration of an analyte 128 in sampling volume 126. The device of FIG. 7 has the same internal configuration as the device 100 described above with respect to FIG. 1, and so the individual component parts of the device 400 are not described again here for brevity. The sampling volume 126 can be a region within any container (such as a jar or bottle) capable of retaining the analyte 128 and having at least an optically transparent portion. The spacer 110 has a thickness which is substantially equivalent to the distance the desired region of interest (sampling volume) is away from the contact surface 132.

Depending on the analyte of interest, suitable dopants having emission profiles that overlap absorption bands of the analyte of interest can be selected (as shown, for example by FIG. 2). Whilst the examples above describe the determination of glucose and alcohol levels in a user's blood, the concentration of the analyte 128 in a wider range of substances held in many different kinds of containers can be tested. Using the device 400, it is possible to determine the concentration of analytes in foodstuffs for quality control testing, such as the concentration of glucose in potatoes (by extracting liquid from the potatoes and placing the liquid into an optically transparent container or by simple contact measurement), or an alcohol by volume percentage of wine in a sealed wine bottle. It is also possible to determine whether manuka honey in a sealed jar is an authentic product or not by selecting suitable dopants that have emission profiles that overlap methylglyoxal (MGO) or dihydroxyacetone (DHA) compounds in the manuka honey. The above are provided only as illustrative examples and the skilled person would appreciate that, by selecting appropriate dopants, other analyte concentrations could be determined by device 400.

### Determination of Fluorescence Lifetime Measurements

The following describes in more detail how fluorescence lifetime measurements of the erbium ions are determined.

### Fluorescence Decay Measurements

In the following, we describe how to determine the spontaneous emission rates Γ* and Γ** for a given I(t). Measuring these spontaneous emission rates Γ* and Γ** for cases where analyte concentrations are known provides a way of calibrating the device 100 as discussed above with respect to FIGS. 3a-c.

### Fourier Analysis

First, we have a closer look at a closely-related and well-established technique for the analysis of time signals. Suppose a measurement signal is of the form:

$$f ( t ) = {\sum }_{n - 1}^{N} {c}_{n} exp [ {i}_{{ω}_{n}} t ]$$

For simplicity, let us ignore that this function is complex. Whether this function f(t) is real or complex does not matter much in the following. To determine the frequencies ωₙ in this equation, we multiply the above measurement signal at all times t with an exponential factor exp[−iω t] and then integrate over the time t from 0 to some later time T. This process should be repeated for a large range of frequencies ω. The result is a function which no longer depends on the time t but on the frequency ω:

$$\begin{matrix}F ( ω ) = {Integral}_{0}^{T} dt f ( t ) exp [ - i ω t ] \\ = {\sum }_{n = 1}^{N} {c}_{n} {Integral}_{0}^{T} d t exp [ i ( {ω}_{n} - ω ) t ] \\ = {\sum }_{n = 1}^{N} i {c}_{n} / ( {ω}_{n} - ω ) ( 1 - exp [ i ( {ω}_{n} - ω ) T ] )\end{matrix}$$

Taking a closer look at the above equation, one can immediately see that F(ω) diverges (tends to infinity) whenever the frequency ω comes close to one of the resonance frequencies ωₙ. Hence calculating F(ω) for a given measurement signal f(t) is a good way of finding the resonance frequencies of a system. Since the divergencies occur for any values of the coefficients cₙ, unless these are zero, the cₙ coefficients do not need to be known. The coefficients cₙ do not need to be controlled in an experiment which aims at finding the resonance frequencies ωₙ. Moreover, it does not matter too much what values are chosen for the cut-off time T as long as it is sufficiently different from zero.

What the above paragraph describes is essentially the idea of Fourier transforms, in a nutshell and with some simplifications. These are routinely used to determine the resonance frequencies of signals and have a wide range of applications.

### Generalised Laplace Transforms

Here we are interested in an analogous problem: instead of finding resonance frequencies ωₙ, we are looking for decay rates Γₙ for a measurement signal I(t) of the form:

$$I ( i ) = {\sum }_{n - 1}^{N} {c}_{n} exp [ - {Γ}_{n} t ]$$

This equation describes the de-excitation process of the dopants, namely the intensity of the emitted light as a function of time between light pulses. Instead of finding the resonance frequencies, we are interested in finding the spontaneous emission rates Γₙ of interacting dipole sources. Since we cannot control which dipoles we excite and how, we cannot control the coefficients cₙ in the above equation in experiments.

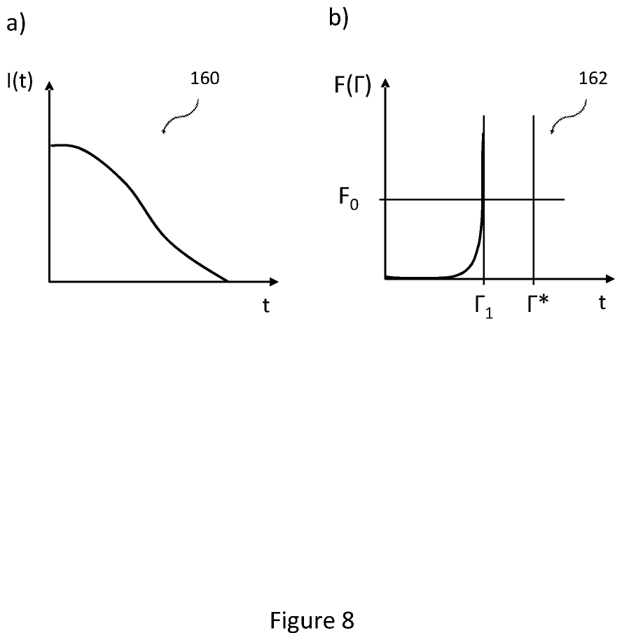

As above, a solution is to multiply the measured fluorescence signal I(t) (as shown by plot 160 in FIG. 8a) with a function of a free parameter Γ and to perform numerical integrations. This should be done such that the resulting function of Γ diverges when Γ comes close to one of the spontaneous decay rates Γₙ. More concretely, we multiply the measured I(t) for all times t with exponentials exp[Γt] and then numerically calculate the integral:

$$\begin{matrix}F ( Γ ) = {Integral}_{0}^{T} dt I ( t ) exp [ Γ t ] \\ = {\sum }_{n = 1}^{N} {c}_{n} {Integral}_{0}^{T} d Γ exp [ - ( {Γ}_{n} - Γ ) t ] \\ = {\sum }_{n = 1}^{N} {c}_{n} / ( {Γ}_{n} - Γ ) ( 1 - exp [ - ( {Γ}_{n} - Γ ) T ] )\end{matrix}$$

As long as the spontaneous decay rate Γ is smaller than all of the spontaneous decay rates Γₙ, all of the above terms in the above equations are well behaved and finite. However, as soon as Γ becomes larger than even one of the Γₙ, the function F(Γ) starts to diverge as shown in plot 162 of FIG. 8b (see solid curve).

The above method allows us to easily identify the smallest spontaneous decay rate Γ₁ involved in the formation of the measurement signal I(t). Using the above-described transformation, which is essentially a Laplace transform (up to some sign choices—i.e., instead of multiplying I(t) by exp[−Γt], here we multiply I(t) instead by exp[Γt] as shown above), therefore allows us to deduce important information about decay processes from I(t). This method works especially well when the signal I(t) is the sum of only a finite number of decaying exponentials. Once one decay rate is known, one can start looking for the next decay rate using only the measured values of I(t) and numerical techniques.

For the problem we consider here, the measurement signal I(t) is in general a combination of a continuum of decaying exponentials, since averages are taken over a large ensemble of emitting dipole sources. In this case, the above transformation of F(Γ) also diverges but not as sharply as in the discrete case (see plot 162 of FIG. 8b). By introducing a cut-off F₀, we can deduce a spontaneous decay rate Γ* which provides information about the approximate size (i.e., an estimate) of the lowest relevant spontaneous decay rate. This then tells us also about the broadness of the distribution p(Γ) and therefore about the strength of the present interactions. This strength is a function of the mean distance between the emitting dipole sources and therefore a direct measure for the concentration of the involved particles.

Here Γ* provides information about the decay processes with the longest lifetimes. In order to also obtain some information about decay processes with relatively short lifetimes, the above method can be applied to the function 1/I(t), i.e., the inverse of the measured fluorescence lifetime signal. The result of this analysis is the spontaneous decay rate which was called Γ** in the previous sections.

Most importantly, we can now deduce information about lifetimes, interaction strengths, etc, without having to be concerned with the size of the coefficients cₙ in the above equations. This approach to data analysis is therefore more robust against errors. Information about target molecule concentrations can now be deduced by comparing the measured values for Γ*_{analyte}, Γ**_{analyte} and Γ_{avg, analyte} with previously calibrated data Γ*_{baseline}, Γ**_{baseline} and Γ_{avg, baseline}.

### Example 1—Proof of Concept Study: Determining Glucose Concentration in Pig Skin

### Method

First, fresh pig ears were collected from an abattoir for use in the experiment. The pig ears had to be collected and processed within 18 hours postmortem, as beyond this time window the skin cells may begin to display pyknosis which indicates the initial stages of tissue necrosis which can lead the chemical and morphological structure of the tissue samples to differ from live tissue to such an extent that it would not be valid in this proof-of-concept study.

The skin of the dorsal side of the pig ears was chosen as it is the most similar to that of human skin. Pig ears have an average stratum corneum (SC) thickness of around 21 μm thick whereas human SC thickness is around 6-19 μm thick. This difference in thickness required the pig ears to be exfoliated using sticky tape in a process called tape stripping. Porcine epidermal thickness is around 72 μm while human epidermal thickness is around 70 μm thick (shoulder). Porcine dermal thickness is around 1.86 mm thick, which corresponds to the thickest human dermal thickness (back) of around 1.8 mm-1.9 mm thick.

The pig ears were then cut into 20 mm×20 mm samples and flash frozen in liquid nitrogen for long term storage. This flash freezing immediately ceases all biological activity without damaging the cell structure or enzymes and prevents degradation of the internal biological structures. In addition, a selection of glucose concentration solutions were prepared ranging from 0 mg/dl to 450 mg/dl in phosphate-buffered saline (PBS). For the testing process, the samples were thawed before soaking in the selection of glucose solutions for 24 hours and each sample was subsequently measured using the device 100. The glucose concentration of each sample was verified with a YSI glucose analyser (widely adopted gold standard laboratory glucose concentration measurement machine).

The gathered fluorescence emission distribution data measured by the device 100 was processed with a machine learning algorithm which analyses 23 or more different features of the fluorescence emission distribution of the dopant (the fluorescence signal obtained from the doped glass 104). Subsequently, using recursive feature selection, the 5 strongest features were used to use to train the algorithm.

### Results

In 2014, the Surveillance Error Grid (SEG) was introduced by a number of authors from academia, industry and regulatory agencies as an accepted measure for assessing the clinical accuracy of blood glucose monitors¹. The main reason for the development of a new error grid was due to changes in clinical and technological standards. In the new error grid, the outcomes of the Diabetes Control and Complications (DCCT) trial² are considered as well as new insulin analogues and higher expectations of the new blood glucose meters on the market. As a result, the Surveillance Error Grid possesses different borders/zones to Clarke and Consensus Error Grids previously relied upon. The borders/zones of the Surveillance Error Grid define a number of different regions, including clinically accurate, clinically acceptable, and three further regions which represent an increasing risk of hypoglycaemia or hyperglycemia. ¹ Klonoff D C, Lias C, Vigersky R, Clarke W, Parkes J L, Sacks D B, Kirkman M S, Kovatchev B; Error Grid Panel. The surveillance error grid. J Diabetes Sci Technol. 2014 July; 8(4):658-72. doi: 10.1177/1932296814539589. Epub 2014 Jun. 13. PMID: 25562886; PMCID: PMC4764212.² Diabetes Control and Complications Trial (DCCT): results of feasibility study. The DCCT Research Group. Diabetes Care. 1987 January-February; 10(1):1-19. doi: 10.2337/diacare.10.1.1. PMID: 2882967.

The measured blood glucose concentrations were plotted on an SEG. As is shown by the table below, 89.1% (or 171) of data points sat within Region A (clinically-accurate) and 6.2% (or 12) data points sat within Region B (clinically-acceptable) of the SEG. This yields an overall accuracy of 95.3% for the device 100 based on the combined results of Region A+B, which is deemed clinically acceptable.

| Risk grade | Number of data points | Percent |
|---|---|---|
| Region A - Clinically | 171 | 89.1% |
| accurate | | |
| Region B - Clinically | 12 | 6.2% |
| acceptable | | |
| Region C | 6 | 3.1% |
| Region D | 3 | 1.6% |
| Region E | 0 | 0% |

1. A device to non-invasively determine a concentration of an analyte in a sampling volume, the device comprising:
a pulsed light source;
a doped glass comprising dopants that are configured to emit fluorescence, the fluorescence having a fluorescence wavelength range that at least partially overlaps an absorption wavelength range of the analyte, the fluorescence having a first temporal fluorescence emission distribution in response to optical excitation by the pulsed light source when the analyte concentration in the sampling volume is zero;
a semi-transparent mirror interposed between the doped glass and the sampling volume, such that the dopants emit fluorescence having a second temporal fluorescence emission distribution that is a function of the concentration of the analyte in the sampling volume; and
a detector configured to measure the second temporal fluorescence emission distribution such that the concentration of the analyte in the sampling volume can be determined based on a change between the first temporal fluorescence emission distribution and the second temporal fluorescence emission distribution.

2. The device of claim 1, wherein the concentration of the analyte in the sampling volume can be determined based on a change between:
a baseline spontaneous emission rate probability distribution based on the first temporal fluorescence emission distribution; and
an analyte-induced spontaneous emission rate probability distribution based on the second temporal fluorescence distribution.

3. The device of claim 2, wherein the change between the baseline spontaneous emission rate probability distribution and the analyte-induced spontaneous emission rate probability distribution can be based on either:
an average of the analyte-induced spontaneous emission rate probability distribution and an average of the baseline spontaneous emission rate probability distribution; or
a broadening of the analyte-induced spontaneous emission rate probability distribution compared to the baseline spontaneous emission rate probability distribution.

4. The device of claim 1, wherein the doped glass and the sampling volume are each located at a distance from the semi-transparent mirror that is greater than a maximum wavelength of the fluorescence wavelength range, for example wherein the distance is in a range of 1 to 100,000 times greater than the maximum wavelength.

5. The device of claim 1, wherein the doped glass and the sampling volume are each located a substantially equal distance from the semi-transparent mirror.

6. The device of claim 1, wherein the device further comprises a spacer interposed between the semi-transparent mirror and the sampling volume.

7. The device of claim 1, wherein a first optical efficiency between the semi-transparent mirror and the sampling volume is different to a second optical efficiency between the semi-transparent mirror and the dopant.

8. The device of claim 7, wherein the device comprises an optical element interposed between either the sampling volume and the semi-transparent mirror or between the semi-transparent mirror and the dopant in order to make the first optical efficiency different to the second optical efficiency.

9. The device of claim 8, wherein the optical element comprises:
a light absorbing component;
a light absorbing layer on a surface of the semi-transparent mirror; or
a roughened surface of an optical component.

10. The device of claim 1, wherein the analyte is a glucose molecule and the dopants are erbium ions.

11. The device of claim 1, wherein the analyte is an alcohol molecule and the dopants are thulium ions.

12. The device of claim 1, wherein the doped glass further comprises a sensitizer, optionally wherein the sensitizer comprises ytterbium ions.

13. The device of claim 1, wherein the analyte is one of a blood analyte or a foodstuff analyte.

14. The device of claim 13, wherein the analyte is a blood analyte and the detector further comprises a contact surface for receiving a skin surface having the blood analyte located at a subcutaneous depth, wherein the semi-transparent mirror is disposed between the contact surface and the doped glass.

15. The device of claim 1, wherein the device further comprises a processor configured to determine the concentration of the analyte in the sampling volume based on the change between the first temporal fluorescence emission distribution and the second temporal fluorescence emission distribution.

16. The device of claim 1, wherein the device further comprises means for transmitting the second temporal fluorescence emission distribution to a remote server for determining the concentration of the analyte in the sampling volume.
